(19) United States
(12) Patent Application Publication
(10) Pub. No.: US 2006/0261242 A1
(43) Pub. Date: Nov. 23, 2006

(54) Image sensor with vertical photo-detector and related method of fabrication

(76) Inventor: Yi-Tae Kim, Suwon-si (KR)

Correspondence Address: VOLENTINE FRANCOS, & WHITT PLLC, ONE FREEDOM SQUARE, 11951 FREEDOM DRIVE SUITE 1260, RESTON, VA, 20190, US

(21) Appl. No.: 11/346,303

(22) Filed: Feb. 3, 2006

(30) Foreign Application Priority Data
Feb. 3, 2005 (KR) 2005-10047

(51) Int. Cl. C12Q 1/68

(52) U.S. Cl. 250/200

(57) ABSTRACT

Disclosed is an image sensor comprising a vertical photo-detector and related method of fabrication. The vertical photo-detector comprises a stacked plurality of photoelectric conversion layers having different optical sensitivities, each respectively separated by a dielectric barrier layer.

### BACKGROUND OF THE INVENTION

1. Field of the Invention

Embodiments of the invention related generally to image sensors. More particularly, embodiments of the invention relate to an image sensor comprising a vertical photo-detector and related methods of fabrication.

This application claims priority to Korean Patent Application 2005-10047 filed on Feb. 03, 2005, the subject matter of which is hereby incorporated by reference.

2. Discussion of Related Art

Image sensors include devices responsive to external light of defined wavelength and adapted to facilitate the display of images related to the light. One form of conventional image sensor employs a scheme wherein an image is displayed in accordance with light received in a defined wavelength or range of wavelengths and thereafter converted in photoelectric conversion elements or regions. The selection or definition of light wavelength may involve the use of one or more filters or filter layers. However, color based light detection and conversion is disadvantageous since it generally requires a large corresponding are for enabling pixels, i.e., the individual constituent photoelectric conversion elements. For instance, if one assumes use of RGB color filters adapted to resolve incident light received by the photoelectric conversion elements into the three primary colors, i.e., red (R), green (G), and blue (B), three corresponding pixels are required to detect the respective colors. If one assumes use of CYGM color filters adapted to resolve incident light into cyan (C), yellow (Y), green (G), and magenta (M), four pixels are required.

One form of conventional image sensor has been proposed which uses a vertical photo-detecting structure adapted to detect incident light and facilitate the display of corresponding color images using a single pixel. This structure departs entirely from the formerly used, horizontal photo-detecting structure in which color filters were arranged in constituent pixel regions.

FIG. 1 is a sectional view of an image sensor having a conventional vertical photo-detector.

Referring to FIG, 1, the image sensor comprises a vertical photo-detector structure in which first-conductivity type diffusion layers, 33, 36, and 40, (hereafter “first diffusion layers”), and the second-conductivity type diffusion layers, 32, 36, and 40, (hereafter “second diffusion layers”), are alternately and vertically stacked in a substrate. The respective diffusion layers in the vertical photo-detector structure comprise depletion regions in which electrons are substantially absent and junctions are formed between the different conductivity boundaries. A pair of the first and second diffusion layers forms a photoelectric conversion layer. The second diffusion layers, 34, 38, and 42, are connected to gate electrodes of source-follower transistors 56r, 56g, and 56b, and source regions of reset transistors 54r, 54g, and 54b. Drain regions of the source-follower transistors, 56r, 56g, and 56b, and the reset transistors, 54r, 54g, and 54b, are connected to a static voltage Vdd. The source-follower transistors, 56r, and 56g, and 56b, are serially connected through respective column selection transistors 58r, 58g, and 58b. Light incident to the vertical photo-detector is communicated in different wavelengths. In accordance with their respective wavelengths, red, green, and blue colors are detected by respective photoelectric conversion layers within the vertical stack of photoelectric conversion layers. In this manner, a single pixel element within an array of pixels forming the image sensor may detect and output information regarding a plurality of colors, respectively detected by a corresponding photoelectric conversion layer in the pixel. This ability offers significant performance advantages, like reduced pixel array size for a given color resolution requirement over the conventional, horizontal photo-detecting structure.

However, the conventional vertical photo-detector suffers from the adverse effects of charge overflow. For example, since the first and second diffusion layers are alternately stacked, charges accumulated in second diffusion layer 38 may be transferred to second diffusion layers 34 and 42 through energy potentials formed in respectively adjacent first diffusion layers 36 and 40. This charge leakage or overflow acts as “color crosstalk” and causes color distortion in ultimately displayed image.

### SUMMARY OF THE INVENTION

Accordingly, embodiments of the invention address the problem of color crosstalk apparent in image sensors comprising conventional, vertical photo-detectors. Integral to the effort of addressing this problem is a requirement that embodiments of the invention also address the problem of charge overflow between photoelectric conversion layers in the vertical photo-detectors.

In one embodiment, the invention provides an image sensor comprising a stacked plurality of photoelectric conversion layers having different optical sensitivities, each respectively separated by a dielectric barrier layer. In a related aspect, at least one of the plurality of photoelectric conversion layers comprises stacked first and second diffusion layers.

In another embodiment, the invention provides an image sensor comprising; a stacked plurality of photoelectric conversion layers having different optical sensitivities, each separated by a dielectric barrier layer, a plurality of source follower transistors, each having a gate electrode respectively connected to one of the plurality of photoelectric conversion layers, a plurality of reset transistors each having a source region respectively connected to one of the photoelectric conversion layers, and a plurality of column selection transistors, each respectively connected in series to one of the plurality of source follower transistors.

In yet another embodiment, the invention provides a method of fabricating an image sensor, comprising; forming a first photoelectric conversion layer on a semiconductor substrate, forming a first dielectric barrier layer on the first photoelectric conversion layer, and forming a second photoelectric conversion layer on the first dielectric barrier layer.

### BRIEF DESCRIPTION OF THE DRAWINGS

The accompanying illustrate several embodiments of the invention and, together with the description, serve to explain principles of the present invention. In the drawings, the thickness of various layers and/or regions may be are exaggerated for clarity. Like reference numerals refer to like elements throughout the specification. In the drawings:

FIG. 1 is a sectional view of an image sensor having a conventional vertical photo-detector;

FIG. 2 is a sectional view of an image sensor having a vertical photo-detector in accordance with an embodiment of the invention;

FIG. 3 is a graph illustrating potentials along depths of the image sensor according to the invention;

FIG. 4 is a sectional view illustrating an image sensor that includes a structure for transferring charges from a photoelectric conversion layer to a source follower transistor;

FIGS. 5A through 5C are sectional views showing processing steps of fabricating an image sensor having the vertical photo-detector in accordance with an embodiment of the invention; and

FIGS. 6A through 6C are sectional views showing processing steps of fabricating an image sensor having the vertical photo-detector in accordance with another embodiment of the invention.

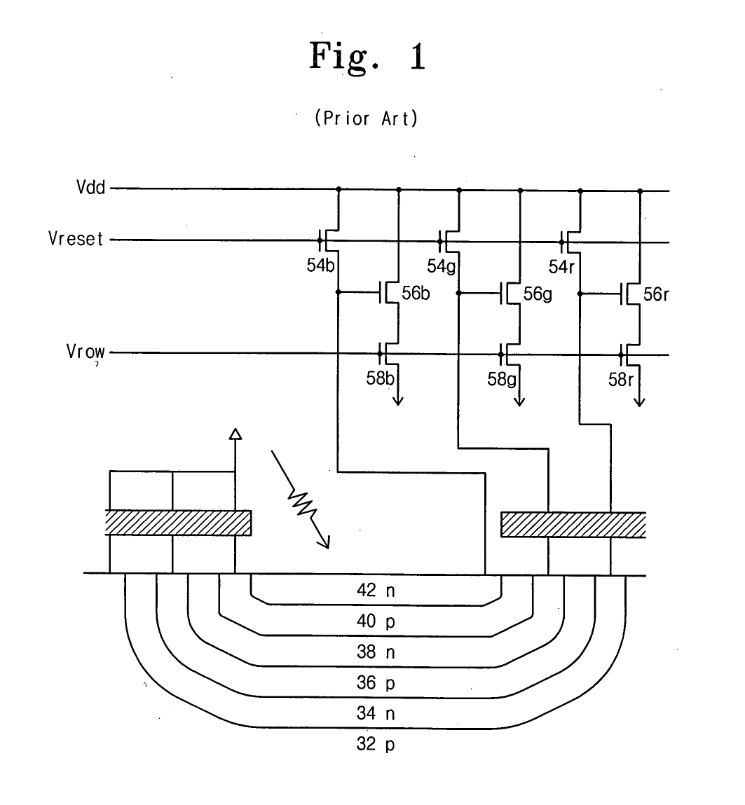

### DESCRIPTION OF EXEMPLARY EMBODIMENTS

Embodiments of the invention will be described with reference to the accompanying drawings. However, the invention may be variously embodied in different forms and should not be constructed as being limited to only the described embodiments. Rather, the embodiments are presented ass teaching examples.

Throughout the description it will be understood that when a layer, region, film, or structure is referred to as being “on” another layer, region, film and/or structure including a substrate, it may be “directly on” the other layer, region, film or structure, or intervening layers may be present.

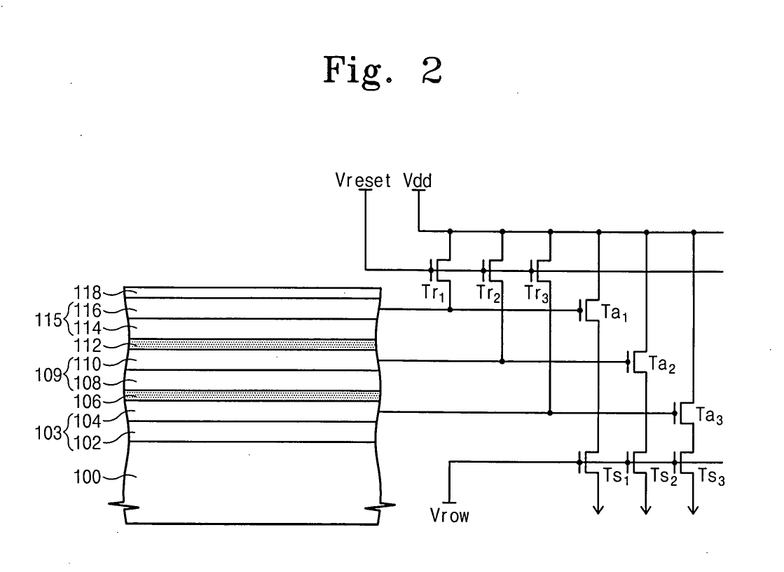

FIG. 2 is a sectional view of an image sensor comprising in relevant portion a vertical photo-detector formed in accordance with an one embodiment of the invention.

Referring to FIG. 2, a first photoelectric conversion layer 103 if formed on a semiconductor substrate 100 and comprised stacked first and second diffusion layers 102 and 104. A second photoelectric conversion layer 109 comprising stacked first and second diffusion layers 108 and 110 is formed on first photoelectric conversion layer 103. A third photoelectric conversion layer 115 comprising stacked first and second diffusion layers 114 and 116 is formed on second photoelectric conversion layer 109.

However, a first dielectric barrier layer 106 is interposed between the first and second photoelectric conversion layers 103 and 109, and a second dielectric barrier layer 112 is interposed between the second and third photoelectric conversion layers 109 and 112. It is preferable to establish appropriate dielectric barrier layer thickness(es) and photoelectric conversion layer doping concentrations such that the respective first and second diffusion regions are fully depleted.

In the illustrated example, the first photoelectric conversion layer 103 is adapted to transforms light having relatively longer wavelength(s) into corresponding electrical signal(s). The second photoelectric conversion layer 109 is adapted to transforms light having relatively mid-range wavelength(s) into corresponding electrical signal(s). The third photoelectric conversion layer 115 is adapted to transforms light having relatively shorter wavelength(s) into corresponding electrical signal(s).

For example, the first photoelectric conversion layer 103 may be configured to convert light wavelengths corresponding to the color red into an electrical signal, the second photoelectric conversion layer 109 may be configured to convert light wavelengths corresponding to the color green into an electrical signal, and the third photoelectric conversion layer may be configured to convert light wavelengths corresponding to the color blue into an electric signal.

In one embodiment the invention, the first and second dielectric barrier layers 106 and 112 are formed from a material having a low absorption coefficient so as not to degrade the overall optical sensitivity of the image sensor. Thus, the first and second dielectric barrier layers 106 and 112 may be variously formed from materials such as silicon oxide, silicon nitride, and silicon oxy-nitride.

In another embodiment, the third photoelectric conversion layer 115 may comprise a pinned diffusion layer 118. The pinned diffusion layer 118 may be formed from a first diffusion layer in order to address the problem of dark current associated generally with photo-detectors.

As further illustrated in the example of FIG. 2, the first diffusion layers, 103, 108, 114, and 118, may be connected to ground. At least the second and third photoelectric conversion layers 109 and 115 may be formed in respective amorphous silicon layers or alternately formed from stacked, single crystal, first and second diffusion layers.

A unit pixel of the image sensor according to the embodiment of the invention illustrated in FIG. 2 comprises reset transistors, Tr1, Tr2, and TR3, source follower transistors, Ta1, Ta2, and Ta3, and column selection transistors Ts1, Ts2, and Ts3. The source follower transistors Ta1˜Ta3 having drain regions connected to static voltage Vdd correspond to each to the photoelectric conversion layers 103, 109, and 115. The gate electrodes of the source follower transistors Ta1˜Ta3 are electrically connected each to the second-conductivity impurity diffusion layers 104, 109, and 116. The reset transistors Tr1˜Tr3 having drain regions connected to static voltage Vdd correspond each to the photoelectric conversion layers 103, 109, and 115. The source regions of the reset transistors Tr1˜Tr3 are electrically connected each to the second-conductivity impurity diffusion layers 104, 110, and 116. The column selection transistors Ts1˜Ts3, having drain regions connected each to the source regions of the source follower transistors Ta1˜Ta3, output signals detected from the photoelectric conversion layers, 103, 109, and 115, in response to a column selection signal Vrow.

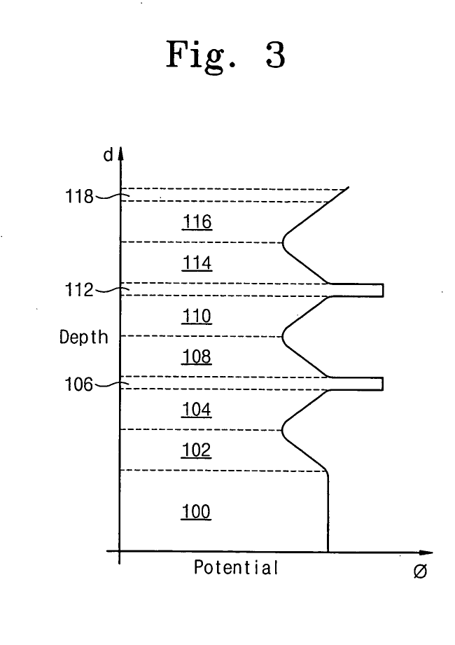

FIG. 3 is a graph illustrating potentials along depths of the exemplary image sensor illustrated in FIG. 2.

Referring to FIGS. 2 and 3, the exemplary image sensor comprises; first through third photoelectric conversion layers, 103, 109, and 115, formed by fully depleted diffusion layers, (i.e., the first and second diffusion layers 103, 108, and 114, and 104, 110, and 116), and the dielectric barrier layers 106 and 112 interposed there between. The respective dielectric layers 106 and 112 have potential barriers higher than those of the pseudo electron bands formed within the respective diffusion layers. Thus, in order for charges overflow to occur from one of photoelectric conversion layer to another, charge would necessarily pass over the potential barrier formed by either the first or second dielectric barrier layers. Therefore, if the potential barriers formed by the first and second dielectric barrier layers 106 and 112 are higher than the potentials of the pseudo electron bands of the respective photoelectric conversion layers, it is possible to fully restrain electron migration between the photoelectric conversion layers 103, 109, and 115.

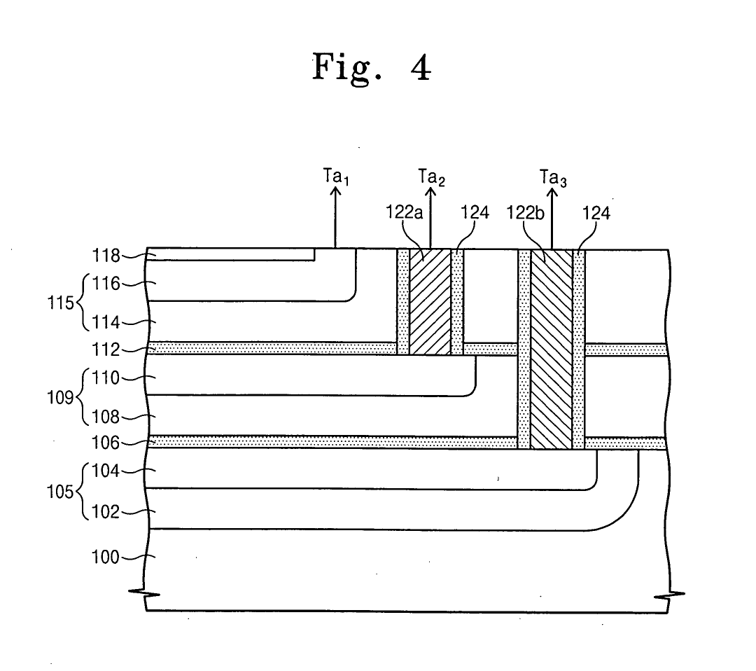

FIG. 4 is a sectional view illustrating one exemplary image sensor comprising a structure adapted to transfers charges from a photoelectric conversion layer to a respective source follower transistor.

Referring to FIG. 4, the first and second diffusion layers, 102 and 104, are formed in semiconductor substrate 100, and the first dielectric barrier layer 106 is deposited on the second diffusion layer 104.

The first and second diffusion layers, 108 and 110, are deposited on first dielectric barrier layer 106. The second dielectric barrier layer 112 is deposited on second diffusion layer 110. The first and second diffusion layers, 114 and 116, are deposited on the second barrier layer 112. A first pinned diffusion layer 118 may be further deposited on second diffusion layer 116. The various diffusion layers, 108, 110, 114, 116, and 118, may be formed from amorphous silicon or single crystalline silicon layer(s).

The second diffusion layer 116 is connected to the gate electrode of the source follower transistor Ta1. The second diffusion layer 110 is connected to the gate electrode of the second source follower transistor Ta2 through a first conductive layer 122a penetrating the (assumed) amorphous silicon layer and the second dielectric barrier layer 112. The second impurity diffusion layer 104 is connected to the third source follower transistor Ta3 through a second conductive layer 122b penetrating the amorphous silicon layer, and the first and second dielectric barrier layers 106 and 112. Insulating sidewalls 124 may be used in conjunction with the first and second conductive layers 122a and 122b.

In the context of the example illustrated in FIG. 4, the first and second conductive layers 122a and 122b penetrate both first diffusion layers 108 and 114. However, were the second diffusion layers 110 and 116 otherwise arranged on the first diffusion layers 108 and 114, the first and second conductive layers 122a and 122b might also penetrate the second diffusion layers 110 and 116, thereby connecting the various diffusion layers in the vertical stack. In this case, insulating sidewalls for first and second conductive layers 122a and 122b would be required to prevent the otherwise connected second diffusion layers 110 and 116 from forming a short circuit.

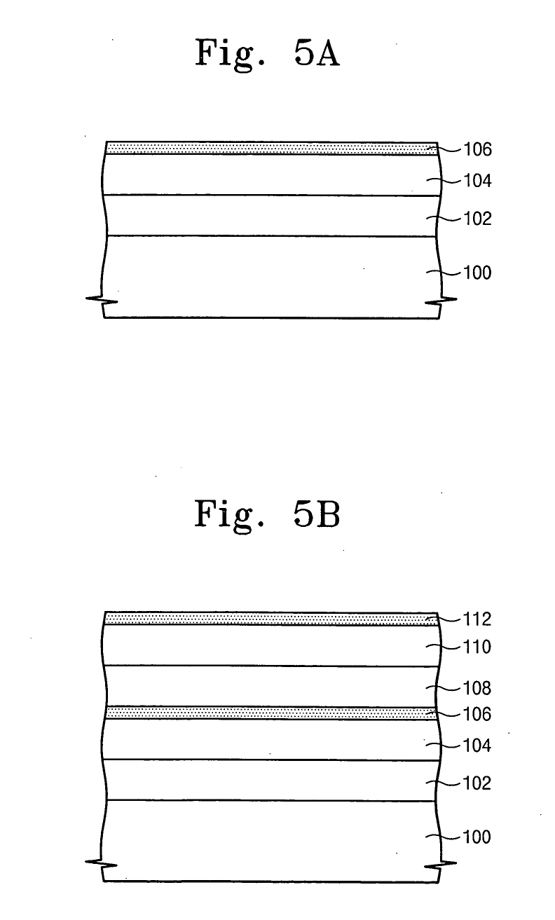

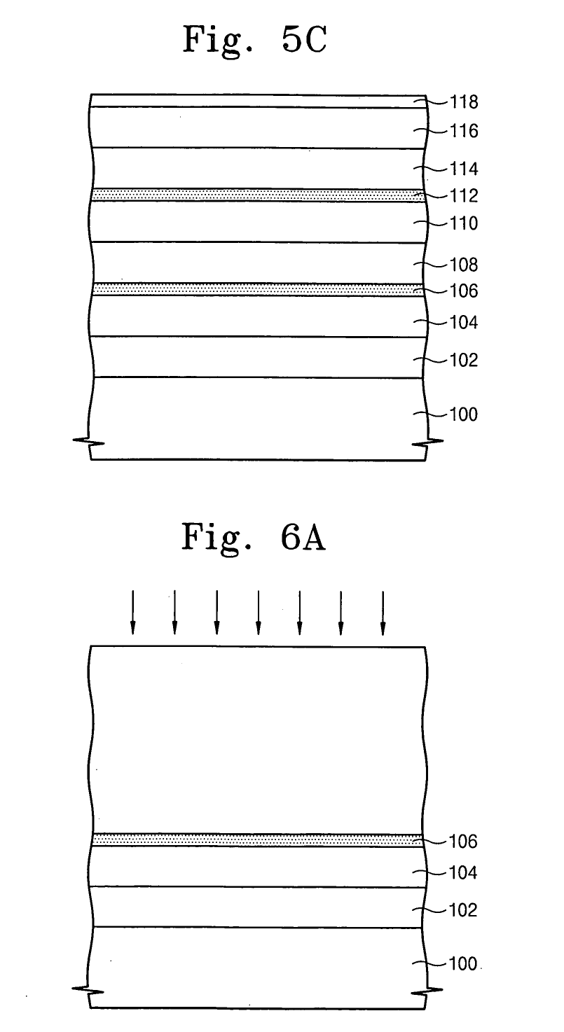

FIGS. 5A through 5C are sectional views showing an exemplary method adapted to the fabrication of an image sensor having the vertical photo-detector in accordance with one embodiment of the invention.

Referring to FIG. 5A, the first and second impurity diffusion layers 102 and 104 are formed on semiconductor substrate 100 by selectively implanting ionic impurities. That is, the first diffusion layer 102 is formed by injecting first-conductivity impurities into the semiconductor substrate 100 at a first depth, and the second diffusion layer 104 is formed by injecting second-conductivity impurities into the semiconductor substrate 100 at a second depth. For instance, the first impurity diffusion layer 102 may be formed by injecting P-type impurities (e.g., boron) into the semiconductor substrate 100, while the second diffusion layer 104 may be formed by injecting N-type impurities, (e.g., phosphorous), into the semiconductor substrate 100.

Here, the semiconductor substrate 100 comprising the first and second impurity diffusion layers 102 and 104 may be formed from a silicon layer epitaxially grown on a silicon substrate having an impurity diffusion layer with doping concentration lower than that of a usual silicon substrate. The first and second impurity diffusion layers 102 and 104 may be completed by impurity diffusion or implantation after growing the epitaxial layer, or completed by doping impurities thereinto while growing the epitaxial layer.

Next, the first dielectric barrier layer 106 is stacked on the second impurity diffusion layer 104. The first dielectric barrier layer 106 may be formed from of a material having a high potential barrier, such as silicon oxide, silicon nitride, or silicon oxy-nitride. The dielectric barrier layer 106 may further be formed from a material having a low absorption coefficient, such as silicon oxide or silicon nitride.

Next, referring to FIG. 5B, after depositing an amorphous polysilicon layer on the first dielectric barrier layer 106, impurities are injected into the amorphous silicon layer to form the first and second diffusion layers 108 and 110. The first diffusion layer 108 may be formed by injecting first-conductivity type impurities into the amorphous polysilicon layer, while the second diffusion layer 110 may be formed by injecting second-conductivity type impurities into the amorphous polysilicon layer. In one embodiment of the invention, it is preferable for the impurity diffusion layers to be doped with low concentration. The first and second impurity diffusion layers, 108 and 110, may be formed by diffusing or injecting impurities after depositing an amorphous silicon layer, or by doping impurities while depositing the amorphous silicon layer.

Thereafter, the second dielectric barrier layer 112 is deposited on the second impurity diffusion layer 110. The second dielectric barrier layer 112 may be formed under the same conditions as the first dielectric barrier layer 106.

Referring to FIG. 5C, another amorphous silicon layer is deposited on the second dielectric barrier layer 112. And then, the first and second diffusion layers, 114 and 116, are formed this amorphous silicon layer. The first diffusion layer 114 may be formed by injecting the first-conductivity type impurities into the amorphous silicon layer, while the second diffusion layer 116 may be formed by injecting the second-conductivity type impurities into the amorphous silicon layer. The pinned diffusion layer 118 may be further formed on the second diffusion layer 116. The pinned diffusion layer 118 may be formed with a thickness less than that of the other diffusion layers. The pinned diffusion layer 118 may be formed by injecting the first-conductivity type impurities. The first and second diffusion layers 114 and 116, and the pinned diffusion layer 118 may be formed by diffusing or injecting impurities after depositing an amorphous silicon layer, or by doping impurities while depositing the amorphous silicon layer.

Although not shown in FIGS. 5A through 5C, the connection structures previously described in relation to FIG. 4 may also be formed.

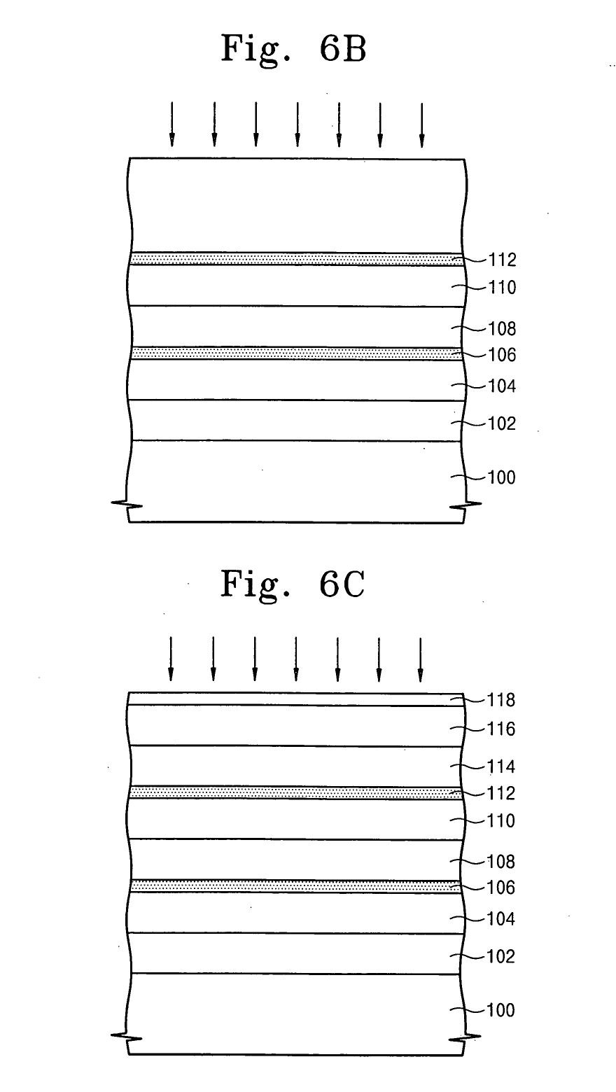

FIGS. 6A through 6C are sectional views showing an exemplary method adapted to the fabrication of an image sensor having the vertical photo-detector in accordance with another embodiment of the invention.

Referring FIG. 6A, the first and second diffusion layers 102 and 104 are formed in the semiconductor substrate 100 by way of implanting ionic impurities. The first impurity layer 102 may be formed by injecting first-conductivity type impurities into the semiconductor substrate 100, while the second diffusion layer 104 may be formed by injecting second-conductivity type impurities into the semiconductor substrate 100. For instance, the first impurity layer 102 may be formed by injecting P-type impurities, (e.g., boron), into the semiconductor substrate 100, while the second diffusion layer 104 may be formed by injecting N-type impurities, (e.g., phosphorous), into the semiconductor substrate 100. Here, the substrate 100 including the first and second diffusion layers 102 and 104 may be formed from an epitaxially grown silicon layer on a silicon substrate, having an impurity diffusion layer with doping concentration lower than that of a usual silicon substrate. The first and second diffusion layers 102 and 104 may be completed by impurity diffusion or implantation after growing the epitaxial layer, or completed by doping impurities thereinto while growing the epitaxial layer.

Next, ions of oxidizing or/and nitrifying agent are injected into the substrate including the first and second diffusion layers 102 and 104, forming the first dielectric barrier layer 106. In other words, the first dielectric barrier layer 106 is formed by injecting oxygen or/and nitrogen ions into the substrate where the first and second impurity diffusion layers 102 and 104 are settled. As a result, the dielectric barrier layer is formed of an alternative one of a silicon oxide film, a silicon nitride film, and a silicon oxy-nitride film on the stacked structure of the first and second diffusion layers 102 and 104. Here, it is preferable for the first dielectric barrier layer 106 to be formed of a silicon oxide film or a silicon nitride film in order to be applicable with the condition of the low absorption coefficient.

Next, referring to FIG. 6B, ionic impurities are injected into the first dielectric barrier layer 106 to form the first and second diffusion layers 108 and 110. The first impurity diffusion layer 108 is formed by injecting first-conductivity type impurities into the amorphous polysilicon layer, while the second diffusion layer 110 may be formed by injecting second-conductivity type impurities into the amorphous polysilicon layer. In one embodiment, it is preferable for the impurity diffusion layers to be doped with low concentration. The second dielectric barrier layer 112 is deposited on the second diffusion layer 110. The second dielectric barrier layer 112 may be also formed by injecting oxygen or/and nitrogen ions into the substrate.

Referring to FIG. 6C, the first and second fifth diffusion layers, 114 and 116, are formed on the second dielectric barrier layer 112. The first diffusion layer 114 may be formed by injecting first-conductivity type impurities into the amorphous silicon layer, while the second diffusion layer 116 may be formed by injecting second-conductivity type impurities into the amorphous silicon layer. The pinned diffusion layer 118 may be further formed on the second diffusion layer 116. The pinned diffusion layer 118 may be formed having a thickness less than that of the other diffusion layers.

As before, the connection structures described in relation to FIG. 4 may also be formed in conjunction with the method illustrated in FIGS. 6A through 6C.

As aforementioned, an image sensor having a vertical photo-detector formed from a plurality (e.g., first, second and third) of photoelectric conversion layers separated respectively by dielectric barrier layers may be sued to prevent problems associated with charge migration. As a result, it is possible to reduce color crosstalk and provide an image sensor having improved color sensitivity.

While the present invention has been described in connection with the forgoing exemplary embodiments, it is not limited to only these embodiments. It will be apparent to those skilled in the art that various substitutions, modifications and changes may be thereto without departing from the scope of the invention which is defined by the following claims.

What is claimed is:

1. An image sensor comprising:
a stacked plurality of photoelectric conversion layers having different optical sensitivities, each respectively separated by a dielectric barrier layer.

2. The image sensor of claim 1, wherein the one of the plurality of photoelectric conversion layers comprises a doped amorphous silicon layer.

3. The image sensor of claim 1, wherein each one of the plurality of photoelectric conversion layers comprises stacked first and second diffusion layers.

4. The image sensor of claim 3, wherein the stacked first and second diffusion layers are formed from one or more amorphous silicon layers doped with first and second conductivity type impurities.

5. The image sensor of claim 3, further comprising a pinned diffusion layer formed on one of the plurality of photoelectric conversion layers.

6. The image sensor of claim 1, wherein the dielectric barrier layer is formed from a material having a potential barrier having than the respective photoelectric conversion layers.

7. The image sensor of claim 6, wherein the dielectric barrier layer is formed from silicon oxide, silicon nitride, or silicon oxy-nitride.

8. An image sensor comprising:
a stacked plurality of photoelectric conversion layers having different optical sensitivities, each separated by a dielectric barrier layer;
a plurality of source follower transistors, each having a gate electrode respectively connected to one of the plurality of photoelectric conversion layers;
a plurality of reset transistors each having a source region respectively connected to one of the photoelectric conversion layers; and
a plurality of column selection transistors, each respectively connected in series to one of the plurality of source follower transistors.

9. The image sensor of claim 8, wherein at least one of the plurality of photoelectric conversion layers is formed from a doped amorphous silicon layer.

10. The image sensor of claim 8, wherein each one of the plurality of photoelectric conversion layers is formed from stacked first and second diffusion layers.

11. The image sensor of claim 9, wherein the amorphous silicon layer is doped with first and second-conductivity type impurities.

12. The image sensor of claim 10, further comprising; a pinned diffusion layer formed on one of the plurality of photoelectric conversion layers.

13. The image sensor as set forth in claim 8, wherein the dielectric barrier layer is formed from a material having a potential barrier higher than the photoelectric conversion layers.

14. The image sensor as set forth in claim 13, wherein the dielectric barrier layer is formed from silicon oxide, silicon nitride, or silicon oxy-nitride.

15. A method of fabricating an image sensor, comprising:
forming a first photoelectric conversion layer on a semiconductor substrate;
forming a first dielectric barrier layer on the first photoelectric conversion layer; and,
forming a second photoelectric conversion layer on the first dielectric barrier layer.

16. The method of claim 15, wherein forming the first photoelectric conversion layer comprises; injecting impurities of first and second-conductivity types into the semiconductor substrate to form stacked first and second diffusion layers.

17. The method of claim 16, wherein forming the second photoelectric conversion layer comprises:
forming an amorphous silicon layer on the first dielectric barrier layer; and,
injecting impurities of first and second-conductivity types into the amorphous silicon layer to form stacked first and second diffusion layers.

18. The method of claim 15, further comprising;
forming a second dielectric barrier layer on the second photoelectric conversion layer; and
forming a third photoelectric conversion layer on the second dielectric barrier layer.

19. The method of claim 15, wherein the dielectric barrier layer is formed of a material having a potential barrier than the first or second photoelectric conversion layers.

20. The method of claim 19, wherein the dielectric barrier layer is formed from silicon oxide, silicon nitride, or silicon oxy-nitride.
